(19) United States
(12) Patent Application Publication
(10) Pub. No.: US 2014/0080789 A1
(43) Pub. Date: Mar. 20, 2014

(54) Method of Treating Diabetes, Glucose Intolerance, Insulin Resistance and/or Diabetes-Induced Diastolic Dysfunction

(71) Applicant: Samuel C. DUDLEY, JR., Providence, RI (US)

(72) Inventor: Samuel C. DUDLEY, JR., Providence, RI (US)

(73) Assignees: The Board of Trustees of the University of Illinois, Urbana, IL (US); U.S. Department of Veterans Affairs, Washington, DC (US)

(21) Appl. No.: 14/083,841

(22) Filed: Nov. 19, 2013

Related U.S. Application Data
(63) Continuation-in-part of application No. 13/658,943, filed on Oct. 24, 2012.
Application No. 13/658,943 is a continuation-in-part of application No. 11/895,883, filed on Aug. 27, 2007.
(60) Provisional application No. 61/552,500, filed on Oct. 28, 2011.
(60) Provisional application No. 60/840,368, filed on Aug. 25, 2006.
(60) Provisional application No. 61/728,302, filed on Nov. 20, 2012.

(51) Int. Cl. A61K 31/675

(52) U.S. Cl. CPC A61K 31/675
USPC 514/89

(57) ABSTRACT

A method of treating, reversing, or ameliorating type 2 diabetes (DM) includes administering to a host in need thereof a therapeutically effective amount of a mitochondria-targeted antioxidant, such as mito-TEMPO (2-(2,2,6,6-Tetramethylpiperidin-1-oxyl-4-ylamino)-2-oxoethyl)triphenylphosphonium chloride).

### CROSS-REFERENCE TO RELATED APPLICATIONS

This is a continuation-in-part (CIP) application of U.S. application Ser. No. 13/658,943, filed Oct. 24, 2012, which claims the priority benefit of U.S. Provisional Patent Application Ser. No. 61/552,500, filed Oct. 28, 2011, and which is a continuation-in-part (CIP) application of U.S. application Ser. No. 11/895,883, filed Aug. 27, 2007, which claims the priority benefit of U.S. Provisional Patent Application Ser. No. 60/840,368, filed Aug. 25, 2006, all are hereby incorporated herein in their entirety by reference. This application further claims the priority benefit of U.S. Provisional Patent Application Ser. No. 61/728,302, filed Nov. 20, 2012, which is also hereby incorporated herein in its entirety by reference.

### STATEMENT REGARDING FEDERALLY SPONSORED RESEARCH OR DEVELOPMENT

The work leading to the present invention was supported by National Institutes of Health grants P01 HL058000, R01 HL1024025, R01 HL106592, Veterans Administration Merit Award, and R41HL112355 to SCD. The U.S. Government therefore has certain rights in the invention.

### FIELD AND BACKGROUND OF THE INVENTION

The present invention is generally directed to cardiac and diabetes treatment and therapy, and more particularly to a method of treating, preventing, reversing, or ameliorating diabetes, obesity, glucose intolerance, insulin resistance, and diabetes-induced diastolic dysfunction.

Heart Failure (HF) is a major and growing public health problem in the United States affecting ˜5 million patients in this country. More than 550,000 patients are diagnosed with new heart failure each year. The disorder is the primary reason for 12 to 15 million office visits and 6.5 million hospital days each year. For many years, the syndrome of heart failure was considered to be synonymous with diminished contractility or reduced ejection fraction (EF). Over the past several years, however, there has been a growing appreciation that a large number of patients with HF have a relatively normal EF or preserved EF. Multiple studies have confirmed that up to half of all HF results from diastolic dysfunction with a preserved EF. The prevalence of diastolic dysfunction (DD) with preserved EF is increasing and virtually all patients with heart failure symptoms, including systolic heart failure, have diastolic dysfunction. In patients >70 years with diastolic dysfunction, the 5-year mortality rate and 1-year hospitalization rates are 50% and 50%, respectively. Epidemiological risk factors for DD include age, hypertension, and diabetes mellitus (DM). DD is observed in about 40% of patients with DM. Several lines of evidence indicate that left ventricular DD represents the earliest preclinical manifestation of diabetic cardiomyopathy and that this can progress to symptomatic HF. Recent studies have demonstrated up to 60% of asymptomatic, normotensive patients with type 2 DM have diastolic dysfunction when assessed by conventional echocardiography. Despite the increasing prevalence and ominous implications for life expectancy, there are currently no approved therapies to slow the progression of DD linked to DM, in part, because of our overall poor understanding of the mechanisms of the association.

As noted above, half of heart failure patients have normal systolic function, a condition known as heart failure with preserved ejection fraction (HFpEF) (References 1-2). Although the prognosis of these patients is similar to those with heart failure with reduced ejection fraction (HFrEF), there has been no definite treatment because of poor understanding of pathophysiology (Reference 2). Several epidemiologic studies have shown that obesity, type 2 diabetes mellitus (DM) and hypertension are closely associated with HFpEF (References 3-4).

Previously, we have shown that increased cardiomyocyte oxidative stress causes diastolic dysfunction as a result of S-glutathinylation of the myofibrillar protein cardiac myosin binding protein-C (cMyBP-C) (Reference 5). Hypertension-induced diastolic dysfunction and S-glutathionylation of cMyBP-C can be prevented by BH₄ treatment (Reference 6). Furthermore, we have shown that other conditions that increase cardiac oxidative stress, such as angiotensin II exposure and mitochondrial manganese superoxide dismutase depletion, also lead to diastolic dysfunction (Reference 5). Mitochondrial oxidative stress is the major underlying pathophysiology for type 2 DM and its complications (References 7-8). In humans and animal models, obesity, insulin resistance, and type 2 DM are associated with an altered cardiac metabolism characterized by an increased production of free radicals or impaired antioxidant defenses (References 7-9). Therefore, we hypothesized that DM causes diastolic dysfunction by increasing mitochondrial oxidative stress leading to S-glutathionylation of cMYBP-C. Here, we tested this by reversing increased mitochondrial reactive oxygen species production (ROS) using a mitochondria-targeted antioxidant (mito-TEMPO).

### Aspects of the Invention

The present disclosure is directed to various aspects of the present invention.

One aspect of the present invention is demonstration that diabetes mellitus (DM) causes diastolic dysfunction.

Another aspect of the present invention is demonstration that diabetes mellitus (DM) causes diastolic dysfunction by increasing mitochondrial oxidative stress leading to S-glutathionylation of cMYBP-C.

Another aspect of the present invention is demonstration that DM and the associated disorders can be ameliorated by reversing increased mitochondrial reactive oxygen species production (ROS) using a mitochondria-targeted antioxidant, such as mito-TEMPO (2-(2,2,6,6-Tetramethylpiperidin-1-oxyl-4-ylamino)-2-oxoethyl)triphenylphosphonium chloride).

Another aspect of the present invention is demonstration that a mitochondria-targeted antioxidant, such as mito-TEMPO, ameliorates or prevents diastolic dysfunction in obese subjects, or those having impaired glucose tolerance or with insulin resistance.

Another aspect of the present invention is demonstration that a mitochondria-targeted antioxidant, such as mito-TEMPO, ameliorates or prevents diabetes-induced diastolic dysfunction.

Another aspect of the present invention is demonstration that disorders, such as obesity, glucose intolerance, and insulin resistance, can be ameliorated or reversed by using a mitochondria-targeted antioxidant, such as mito-TEMPO.

Another aspect of the present invention is demonstration that a mitochondria-targeted antioxidant, such as mito-TEMPO, reduces or prevents oxidant-mediated S-glutathionylation of myosin binding protein-C (MyBP-C), in obese or diabetic conditions.

Another aspect of the present invention is demonstration that by depressing or reducing S-glutathionylation of myosin binding protein-C (MyBP-C) in obese or diabetic conditions, a mitochondria-targeted antioxidant, such as mito-TEMPO ameliorates or prevents diastolic dysfunction.

A method of treating, reversing, or ameliorating type 2 diabetes (DM) includes administering to a host in need thereof a therapeutically effective amount of a mitochondria-targeted antioxidant, such as mito-TEMPO.

A method of treating, reversing, or ameliorating insulin resistance includes administering to a host in need thereof a therapeutically effective amount of a mitochondria-targeted antioxidant, such as mito-TEMPO.

A method of treating, reversing, or ameliorating glucose intolerance includes administering to a host in need thereof a therapeutically effective amount of a mitochondria-targeted antioxidant, such as mito-TEMPO.

A method of treating, reversing, or ameliorating diabetes-induced diastolic dysfunction includes reducing S-glutathionylated myosin binding protein-C (MyBP-C) level by administering to a host in need thereof a therapeutically effective amount of a mitochondria-targeted antioxidant, such as mito-TEMPO.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

One of the above and other aspects, novel features and advantages of the present invention will become apparent from the following detailed description of the non-limiting preferred embodiment(s) of invention, illustrated in the accompanying drawings, wherein:

FIG. 1 illustrates Intra-peritoneal glucose tolerance test. Serial measurements of serum glucose level following intra-peritoneal challenge of glucose at 2 gram/kg of body weight. N=5 to 7 in each group. Data is mean±SEM. * denotes p value less than 0.05.

FIGS. 2A-F illustrate myocardial tagged magnetic resonance imaging. FIG. 2A illustrates myocardial tagging images were obtained from end-systole (a) to end-diastole (b) every 5 msec. Intersections of each tagging lines were traced manually (c). Endocardium and epicardium were contoured by B-spline method (d). By dividing each grid line into two triangles, harmonic phase analyses were performed in each triangle to calculate circumferential and radial strain (e). FIG. 2B illustrates MRI measurement of circumferential strain (Ecc) rate during the rapid filling phase. FIG. 2C illustrates regional and global Ecc rate following 4 weeks of HFD. FIG. 2D illustrates regional and global Ecc rate following 8 weeks of HFD. N=5-7 in each group. FIG. 2E illustrates longitudinal assessment of Ecc rate. N=4 in each group. FIG. 2F illustrates longitudinal assessment of LV mass. N=4 in each group. * denotes p<0.05. ** does p<0.01.

FIGS. 3A-H illustrate HFD develop diastolic dysfunction without fibrosis or AGE. FIG. 3A illustrates echocardiographic measurement of mitral inflow (a) from the apical four chamber view. Ratio of the early rapid filling phase (E) and late atrial kick flow and active filling phase (A), E/A in pulse-wave Doppler. FIG. 3B illustrates tissue Doppler measurement shown in ratio of diastolic early and late phase, E′/A′. FIG. 3C illustrates ratio of E/E′. FIGS. 3D and 3E illustrate representative images of pulse-wave Doppler and tissue Doppler in HFD group. FIG. 3F illustrates representative invasive hemodynamic measurement of multiple loops of end-diastolic pressure-volume relationship (EDPVR). FIG. 3G illustrates trichrome staining of myocardium. FIG. 3H illustrates Immunohistochemistry for AGE. N=3 in each group.

FIGS. 4A-B illustrate comparison of non-invasive CMR, echocardiography and invasive hemodynamic measurement in diastolic dysfunction. FIG. 4A illustrates linear correlation between MRI measurement and invasive hemodynamic measure (left). Corresponding bland-altman plot (right). FIG. 4B illustrates linear correlation between tissue Doppler and invasive hemodynamic measures (left). Corresponding bland-altman plot (right).

FIGS. 5A-F illustrate mito-TEMPO effect on insulin resistance and diastolic dysfunction. FIG. 5A illustrates body weight were measured at each group. Eight weeks of HFD group were induced the significant obesity and the body weight of the HFD is reduced to the level of control in mito-TEMPO treated group. FIG. 5B illustrates glucose tolerance test. With mito-TEMPO administration for 8 weeks *: p<0.05 compared with the control and HFD+mito-TEMPO groups by LSD post HOC. FIG. 5C illustrates fasting serum glucose levels. Mice were fasted for 6 hours and measured serum glucose levels. FIG. 5D illustrates fasting serum insulin level. Six hours fasting serum insulin level was significantly higher in HFD group than the age matched control. With the treatment with mito-TEMPO for 8 weeks, the fasting insulin level was significantly reduced to the level of the control. *:p<0.05 by ANOVA. FIG. 5E illustrates MRI measurement of diastolic function. FIG. 5F illustrates MRI measurement of LV mass. Mito-TEMPO effect on diastolic function and LVH Data represent mean±SEM. N=7 to 10 each group. * denotes p<0.05.

FIGS. 6A-E illustrate effect of mito-TEMPO in mechanical contraction and relaxation properties of HFD cardiomyocytes. FIG. 6A illustrates diastolic sarcomere length. FIG. 6B illustrates relaxation constant, tau. FIG. 6C illustrates time to 50% relaxation. FIG. 6D illustrates relaxation velocity. FIG. 6E illustrates fractional shortening was comparable in all groups. N=5 to 7 in each group. Data represent mean±SEM. * denotes p<0.05. ** does p<0.01.

FIGS. 7A-D illustrate mito-TEMPO reduced mitochondrial oxidative stress in HFD mice. FIG. 7A illustrates mitochondrial ROS measured by flowcytometry. MitoSOX were stained for 15 min in each group of isolated cardiomyocytes. Singlet rod shape of cardiomyocytes (10,000 cells) were gated and counted the intensity of mitoSOX. FIG. 7B illustrates quantitative data of each histogram from mean fluorescence of mitoSOX intensity. FIG. 7C illustrates histogram of DHE intensity. FIG. 7D illustrates quantitative data of each histogram from mean fluorescence of DHE intensity. N=3-4 in each group. * denotes p<0.05.

FIGS. 8A-B illustrate electron microscopy images to show mitochondrial morphology impairment. FIG. 8A illustrates control group (a and d), HFD (b and e) and mito-TEMPO (c and f) treated HFD. Magnification 5,780X (a, b and c) and 116,000X (d, e and f). Bar indicate 10 μm. FIG. 8B illustrates quantification of mitochondrial areas on a same magnification. N=3 in each group.

FIGS. 9A-C illustrate non-specific protein nitrotyrosylation measurement. FIG. 9A illustrates immunoblotting against nitrotyrosiylation. Each proteins were normalized by actin. FIGS. 9B and 9C illustrate quantification of actin normalized nitrotyrosylation at 30 kDa and 50 kDa, respectively. Both of 30 KDa and 50 KDa proteins were elevated nitrotyrosylation and recovered by mito-TEMPO treatment. N=4-8 in each group. * denotes p<0.05.

FIGS. 10A-C illustrate redox-sensitive S-gluatathionylation of cMyBP-C in myofilament post-translational modification. FIG. 10A illustrates immunoblotting of S-glutathionylated cMyBP-C on purified myofilament fractions. Purified myofilament proteins were separated in 12% SDS-PAGE. FIGS. 10B and 10C illustrate quantitative densitometry graphs. cMyBPC (114 kDa) were normalized by total protein (FIG. 10B) and actin (FIG. 10C) N=4-8 in each group. * denotes p<0.05. ** does p<0.01.

### DETAILED DESCRIPTION OF THE PREFERRED EMBODIMENT(S) OF THE INVENTION

Recent evidence has shown a close relationship of mitochondrial dysfunction with the type 2 DM and its significant risk factors obesity and insulin resistance. Also, obesity and type 2 DM are closed associated with heart failure with preserved ejection fraction (HFₚEF). In patients with the type 2 DM, mitochondrial oxidative stress can lead to mitochondrial morphologic change, reduction in the functional capacity and mtDNA mutations, and we have shown that cardiomyocyte oxidative stress leads to hypertension-associated diastolic dysfunction. These mitochondrial alterations may have pathogenic effect in the organs central to glucose and insulin metabolism. In pancreatic beta cells, appropriate insulin secretion in response to the serum glucose level is dependent on adenosine triphosphate (ATP), which is mainly generated by mitochondria. In skeletal muscles and the liver, when mitochondrial functional capacity is reduced, intracellular fats may accumulate, leading to insulin resistance and derangements in insulin signal transduction. In adipocytes, reduced mitochondrial functional capacity may lead to impaired thermogenesis and energy expenditure, resulting to obesity and insulin resistance.

Therefore, we hypothesized that chronic overload of energy substrate and intracellular fat accumulation, elevated PKC delta, mitochondrial oxidative stress generate a vicious cycle in skeletal muscles and the liver, leading to obesity, insulin resistance. In the brown fat tissue, this cycle results in impaired thermogenesis, which in return, can generate more mitochondrial oxidative stress. In addition, this vicious cycle may overexpress an uncoupling protein (UCP)-2 in pancreatic beta cells, leading to further negative feedback to mitochondrial functional capacity and insulin secretory dysfunction. Our preliminary data (below) have shown that C57BL6/J mice begin to develop significant obesity, insulin resistance and glucose intolerance after 3 weeks of high fat diet (HFD), compared with the age matched, low fat diet control. These metabolic derangements were accompanied by mitochondrial morphologic changes, proliferation and reduction in functional capacity. Finally, these pathologic derangements were prevented by systemic administration of mitochondrial antioxidant, mito-TEMPO. High fat diet-induced obese (DIO) mice were chosen for this proposal because of clinical relevance in developing obesity and insulin resistance.

### METHODS

### High Fat Diet-Induced Obese and Insulin Resistant Mouse

Animal care and interventions were provided in accordance with the National Institute of Health (NIH) Guide for the Care and Use of Experimental Animals, and all animal protocols were approved by the Institutional Animal Care and Use Committee of the University of Illinois at Chicago. Six-week old male C57BL6/J mice were purchased from Jackson laboratory (Bar Harbor, Mass.). The HFD (high fat diet) group was fed 60 kcal % fat diet (Research Diets. Inc, New Brunswick, N.J.) for eight weeks. The age matched control group was fed normal chow (Harlan, Indianapolis, Ind.) for eight weeks. A subgroup of HFD mice was treated with mito-TEMPO while these mice continued a HFD. Mito-TEMPO was administered at 0.5 mg/kg twice a day intraperitoneally. Following eight weeks of HFD, mice underwent myocardial tagged magnetic resonance imaging (MRI) and sacrifice to harvest tissues for ex vivo studies.

### Measurement of Plasma Glucose and Insulin

Serum glucose levels were measured by a glucose meter (ACCU-CHEK, Roche Applied Science, Indianapolis, Ind.) after drawing blood from the tail vein. After sacrifice, blood was also collected by cardiac puncture and centrifuged to separate plasma. Plasma insulin level was measured using an enzyme-linked immunosorbent assay kit (Millipore, Billerica, Mass.).

### Myocardial Tagged Magnetic Resonance Imaging

Following general anesthesia using 1-1.5% isoflurane, mouse myocardial tagged magnetic resonance imaging (CMR) was performed on a 600-MHz Bruker Avance console (Bruker Biospin, Billerica, Mass.) equipped with an actively shielded 14.1-T, 89-mm-bore vertical magnet and a 1000 mT/m, 110 μs rise-time microimaging gradient system (Reference 10). Three 1 mm slices of short axis cine images were acquired covering the entire left ventricle (LV) with cardiac and respiratory gating. From these cine images, LV volume and mass were calculated by contouring the endo- and epicardium using Osirix Imaging software (Bernex, Switzerland). In addition, these cine images provide an accurate timing for end-systole, which was defined as the smallest LV cavity volume. A myocardial tagged mid-ventricular short axis image was obtained using cardiac and respiratory-gated spatial modulation of magnetization (SPAMM) sequence (Reference 11). After tagging-grid generation, multiple tagged-images were acquired from end systole throughout LV diastole with a temporal resolution of 5 ms. Image analysis was processed by Matlab (MathWorks, Natick, Mass.). Serial motions of the tagging grid were tracked manually. Deformed tagging square-like elements were divided into two adjacent triangles for homogeneous strain calculations from the reference time point, end-systole (References 12-13). Maximal circumferential strain (Ecc) rate during the rapid filling phase was calculated to assess diastolic function (Reference 14-15).

### Doppler Echocardiography

Mitral inflow velocity and longitudinal tissue velocity of mitral anterior annulus were assessed using Vevo 770 high-resolution in vivo imaging system (Visual Sonics, Toronto, Canada) (Reference 6). During the procedure, mice were anesthetized with 1-1.5% isoflurane until a heart rate of 350-390 beats/min was achieved because measures of diastolic function are sensitive to heart rate and loading conditions.

### Invasive Hemodynamic Measurement

Under the general anesthesia using 1-1.5% isoflurane, a pressure-volume (PV) catheter was inserted into the right common carotid artery and advanced into LV. Inferior vena cava occlusion was performed via a diaphragm incision. Following calibration of volume and parallel conductance, baseline hemodynamic measurements were obtained. Multiple pressure volume loops were acquired during compression of inferior vena cava. End-diastolic pressure volume relationship was generated using linear regression (Reference 5).

### Assessment of Mechanical Properties of Cardiomyocytes

Hearts were excised from anesthetized mice and perfused with buffer (in mmol/L: NaCl 113, KCl 4.7, Na₂HPO₄ 0.6, KH₂PO₄ 0.6, MgSO₄ 1.2, Phenol Red 0.032, NaHCO₃ 12, KHCO₃ 10, HEPES 10, Taurine 30, 2-3-butanedione monoxime 10) and digested with collagenase II (Worthington Biochemical Co. Lakewood, N.J.) for 10 min at 37° C. Following washing with control buffers (in mmol/L: NaCl 133.5, KCl 4, Na₂HPO₄ 1.2, HEPES 10, MgSO₄ 1.2 and 0.1% Bovine serum albumin) at serially increasing Ca²⁺ concentrations (0.2, 0.5, and 1 mmol/L), cardiomyocytes were suspended in Modified Eagle's Medium with 1% insulin-transferrin-selenium, 0.1% bovine serum albumin, 1% glucose) in a 95% O₂/5% CO₂ incubator at 37° C. (Reference 6).

The mechanical properties of cardiomyocytes were assessed using an IonOptix Myocam System (IonOptix Inc., Milton, Mass.) (Reference 16). Unloaded cardiomyocytes placed on a glass slide for 5 min were imaged with an inverted microscope and perfused with a normal Tyrode's buffer (in mmol/L: 133 NaCl, 5.4 KCl, 5.3 MgCl₂, 0.3 Na₂PO₄, 20 HEPES, 10 glucose, pH 7.4) containing 1.2 mmol/L calcium at 37° C. with a temperature controller. Cardiomyocytes were paced with 10 V, 4 ms square wave pulses at 1.0 Hz, and sarcomere shortening and relengthening were assessed using the following indices: diastolic sarcomere length (SL, μm), peak fractional shortening (FS, %), relaxation time constant τ (calculated as a₀+a₁e^{t/τ} where t=time, s), relengthening time (s), and maximum relaxation velocity (dL/dt, μm/s).

### Measurement of Mitochondrial Oxidative Stress by Flow Cytometry

Following isolation of cardiomyocytes as described above, cells were stained with 1 mM MitoSOX (Invitrogen, Carlsbad, Calif.) and 1 mM dihydroethidium (DHE, Invitrogen, Carlsbad, Calif.). Mean fluorescence intensity was measured using a Cyan ADP analyzer (Beckman Coulter, Brea, Calif.) from 5,000 gate-selected cardiomyocytes in FL2 and FL3 channels respectively. Histograms were generated by FlowJo v7.2.5 (Tree Star, Ashland, Oreg.) (Reference 17).

### Transmission Electron Microscopy

Control, HFD and HFD treated with mito-TEMPO were studied. Tissues were washed with cold phosphate buffered saline (PBS), and fixed with EM Grade glutaraldehyde 4% in 0.1M cacodylate buffer (pH 7.4). Fixed tissues were incubated with osmium tetroxide 1% in cacodylate buffer for 2 h and processed for embedding. Ultra-thin sections were cut 83 nm, placed on 200 mesh copper grids, and stained with uranyl acetate and lead citrate. All materials were purchased from Electron Microscopy Sciences (Hatfield, Pa.). Samples were visualized using a JEM-1220 Jeol transmission electron microscopy (JEM, Peabody, Mass.), and micrographs were taken using a Gatan Digital Micrograph (Gatan Microscopy, Plesanton, Calif.). All microscopy measurements were performed using the University of Illinois Central Microscopy Research Core Facility. Random images (n=5) from each sample were taken for analysis. The area occupied by mitochondria was calculated using Image J (NIH, Bethesda, Md.) (Reference 17).

### Histology and Immunohistochemistry for Advanced Glycation EndProducts (AGE)

Extracted hearts were sectioned along the short axis plane and processed for trichrome staining. The immunohistochemistry was performed for AGE. Following fixing samples in 10% formalin, 8-μm thick sections were blocked for 1 h at room temperature and incubated with anti-AGE antibody (Abcam, Cambridge, Mass.) overnight at 4° C. The slides were reviewed with a Zeiss Axioskop microscope (Carl Zeiss, Inc, Thornwood, N.Y.), and photomicrographs with original magnification x20 were taken from the mid-LV.

Immunoblots for Nitrotyrosine and S-Glutathionylation of cMyBP-C

Proteins were isolated from the frozen ventricles and separated on a 4-12% SDS-PAGE gel and transferred onto a 0.2 μm PVDF membrane. After blocking the membrane with 5% nonfat dry milk, anti-nitrotyrosine mouse monoclonal antibody (Millipore, Billerica, Mass.) was applied to detect this oxidative modification of tyrosine.

Myofibrils were prepared from mouse hearts as described previously (Reference 6). Myofibrils were separated on a 4-12% SDS-PAGE gel and transferred onto a 0.2 μm PVDF membrane. Following blocking the membrane in 5% nonfat dry milk with 2.5 mM NEM for 1 h, anti-glutathione mouse monoclonal primary antibody (Virogen, Watertown, Mass.) was applied to detect for glutathionylation on cMyBP-C. Optical density of the bands was measured with ChemiDoc MP system (Bio-rad, Hercules, Calif.) and analyzed with Quantity One imaging analysis software (Bio-rad, Hercules, Calif.).

### Statistical Analysis

Descriptive statistics include mean and standard error of mean (SEM). Comparison between two groups was performed using student's t-test. For comparison between multiple groups, ANOVA with LSD Post Hoc test was utilized. All data analyses were done by SPSS 16.0 (SPSS Inc, Chicago, Ill.). Significance was assumed when p<0.05.

### Results

### High Fat Diet Induced Metabolic Alterations Similar to Metabolic Syndrome and Type 2 Diabetes

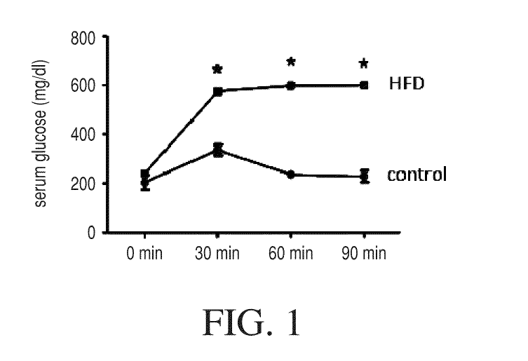

HFD mice developed significant obesity following eight weeks of HFD as shown in Table 1 (below). Random serum glucose was significantly elevated compared to control. Although fasting serum glucose level was similar in both groups, fasting serum insulin level was significantly higher in HFD mice, indicating that HFD mice developed peripheral insulin resistance. As shown in FIG. 1, intra-peritoneal glucose tolerance test showed significant glucose intolerance in the HFD mice compared with the control as follows. 566±20 vs. 375±24 mg/dL at 30 min, 595±6.1 vs. 256±15 mg/dL at 60 min, 597±3.7 vs. 229±23 mg/dL at 90 min, and 509±42 vs. 249±4.4 mg/dL at 120 min following intraperitoneal glucose administration. These results indicated that HFD mice developed metabolic alterations similar to metabolic syndrome and type 2 DM.

### High Fat Diet Induced Diastolic Dysfunction and Left Ventricular Hypertrophy

To evaluate the effect of metabolic alteration on cardiac function and phenotype, ultra-high field cardiac magnetic resonance imaging was performed following eight weeks of HFD. HFD mice showed significant left ventricular hypertrophy compared with the control (Table 2-below). Consequently, end-diastolic volume (EDV) and stroke volume were significantly reduced compared with the control. However, systolic blood pressure was comparable in both groups. Systolic function was preserved as shown by similar ejection fraction (EF) in both groups. In addition, cardiac output (CO) and cardiac index (CI) were also similar in both groups. These results indicated HFD mice developed concentric left ventricular hypertrophy (LVH) with preserved systolic function compared with the control.

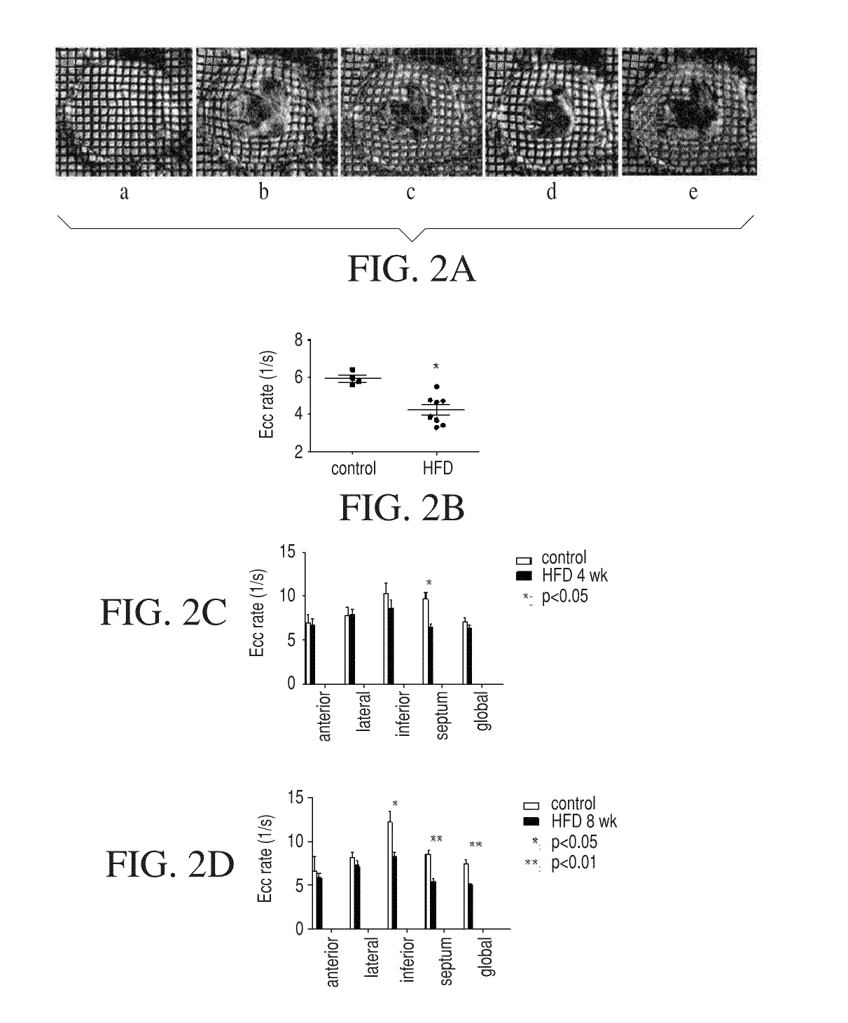

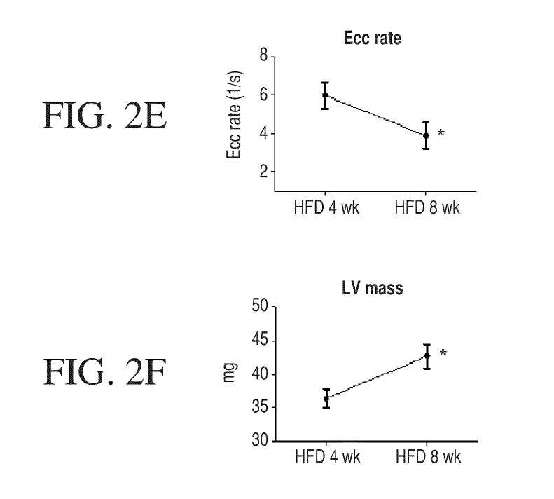

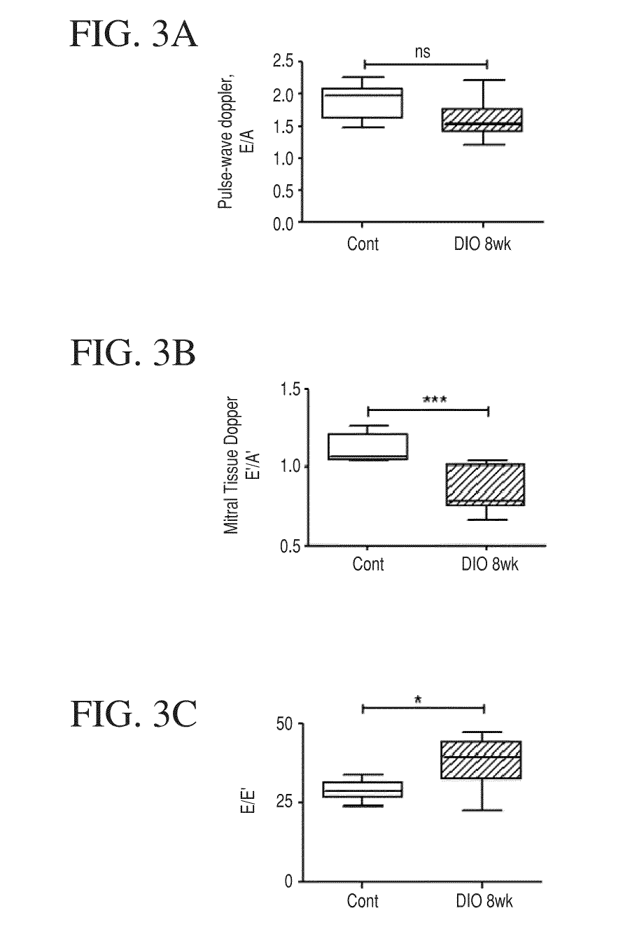

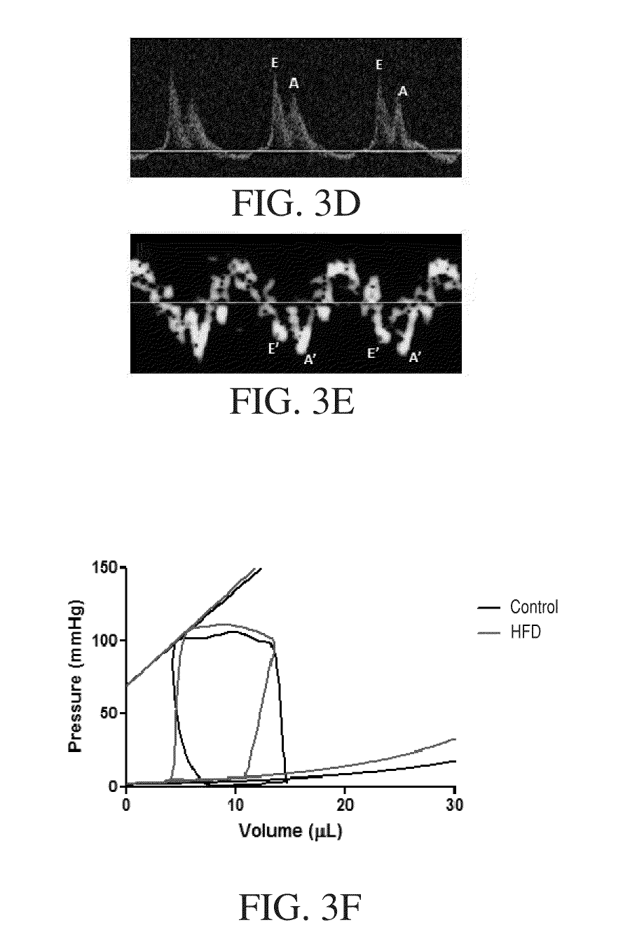

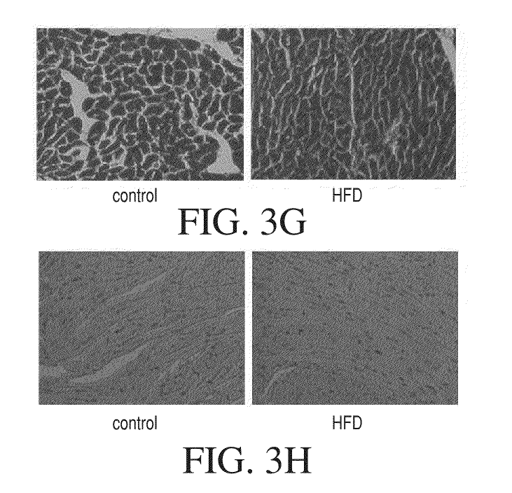

Diastolic function was assessed using three different modalities as demonstrated in FIGS. 2A-F and 3A-H. First, myocardial tissue tagged CMR enabled direct measurement of myocardial strain during diastole shown in FIG. 2A. Ecc rate was significantly reduced during diastole in HFD mice (4.24±0.28 1/s) compared with the control (5.94±0.19 1/s, p<0.05), indicating significant relaxation impairment in HFD mice (FIG. 2B-D). Longitudinal assessment of diastolic function by myocardial tagged CMR revealed progression of diastolic dysfunction with HFD (FIG. 2E). In parallel, LVH also progressed over the period of HFD (FIG. 2F). Second, the first diastolic deflection represents the early rapid filling phase (E), followed by a period of diastasis, and a second late active filling phase (A) due to atrial contraction in pulse-wave Doppler. Tissue Doppler can be represent two negative diastolic early and late (E′; A′) waveforms. Doppler echocardiography revealed significantly increased E/E′ (42±1.4 in the HFD vs. 8.6±3.3 in the control, p<0.01, FIGS. 3A-E). Finally, invasive hemodynamic measurement showed the slope of the end-diastolic pressure volume relationship (EDPVR) was significantly higher in the HFD mice (0.37±0.04) compared with the control (0.21±0.03, p<0.05; FIG. 3F). All three modalities indicated HFD mice developed significant diastolic dysfunction following eight weeks of HFD when compared with control animals.

Trichrome staining demonstrated no evidence of interstitial fibrosis in neither group (FIG. 3G). Immunohistochemistry did not show evidence of deposition of AGE in either group (FIG. 3H).

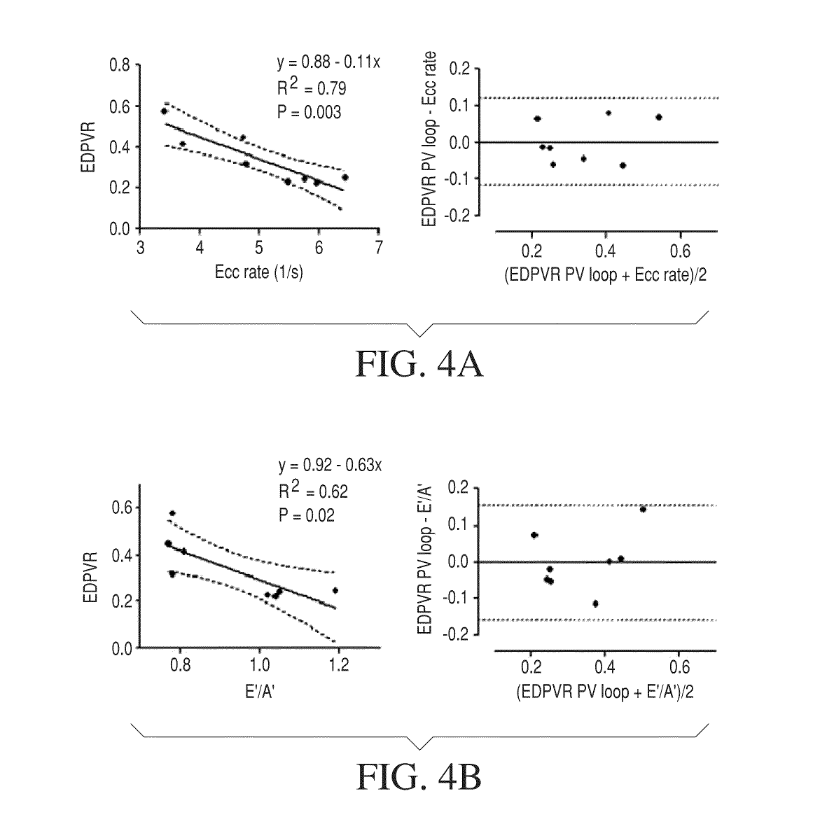

Circumferential ECC rate in CMR was highly correlated in EDPVR in hemodynamics (FIG. 4A), as well as E′/A′ ratio in echocardiography (FIG. 4B).

### Mito-TEMPO Shows Effective Reduction of Mitochondrial Oxidative Stress-Induced Glucose Tolerance and Insulin Resistance in the Type 2 DM

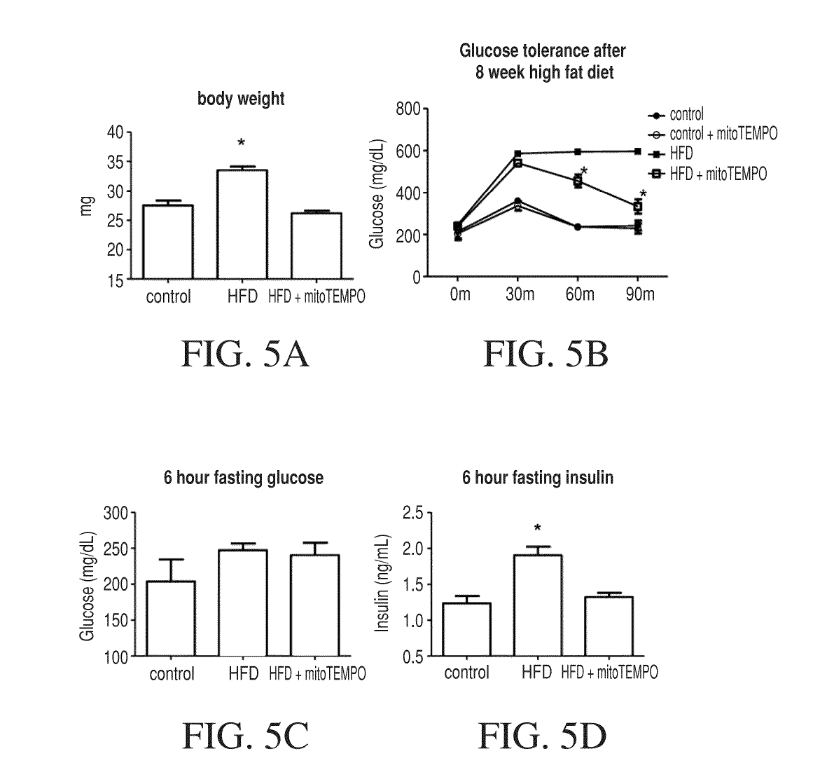

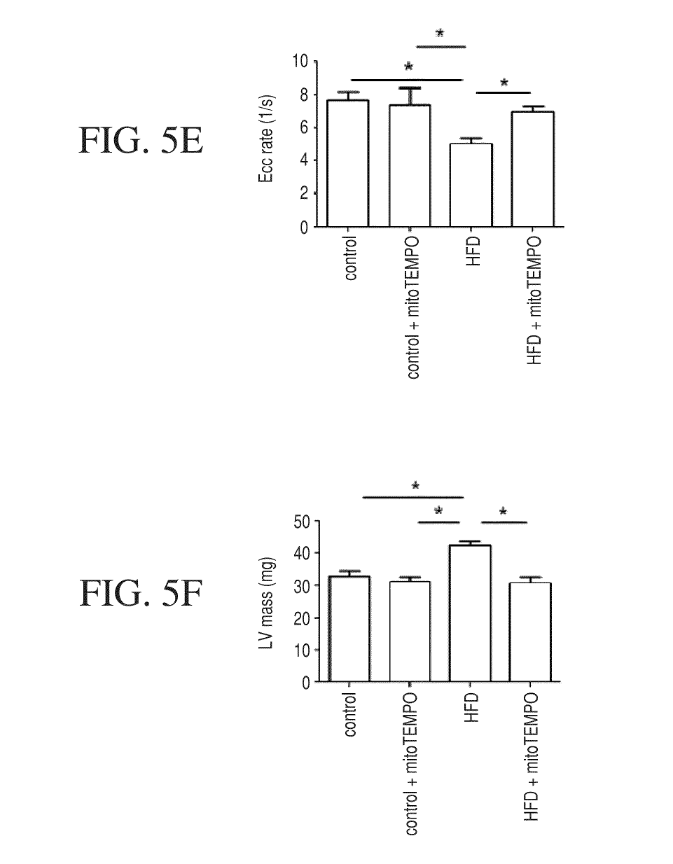

Following 8 weeks of HFD, body weight is increased significantly, compared with the control indicating significant obesity and impaired glucose tolerance shown in HFD group. With mito-TEMPO administration for 8 weeks, the body weight of the HFD is reduced to the level of control. *: p<0.05 compared with the control and HFD with mito-TEMPO treated groups by post HOC. (FIG. 5A). Glucose tolerance test also revealed significantly reduced serum glucose level in the mito-TEMPO treated HFD group at 60 minutes after intra-peritoneal glucose challenge, compared with non-treated HFD group, as demonstrated in FIG. 5B.

Six hours fasting serum glucose levels were compared within the groups, but there were no significant changes by mito-TEMPO treated HFD group (FIG. 5C). However, 6 hours fasting serum insulin level was significantly elevated in HFD group, and dramatically reduced after mito-TEMPO treatment (FIG. 5D). This result indicates that these results suggest mitochondria oxidative stress might mediate obesity and glucose intolerance in the type 2 DM and mito-TEMPO ameliorated insulin resistance to the level of the control, suggesting mitochondrial oxidative stress might cause insulin resistance in the type 2 DM.

### Mito-TEMPO Prevented Diastolic Dysfunction and Left Ventricular Hypertrophy

To assess the role of mitochondrial oxidative stress in developing diastolic dysfunction in type 2 DM, the mitochondrial antioxidant, mito-TEMPO, was administered to a subgroup of HFD mice for eight weeks while these mice continued the HFD. Mito-TEMPO-treated HFD mice showed similar Ecc rate (6.4±0.4 1/s) to the control (7.0±0.6 1/s, p=0.35; FIG. 5E). Mito-TEMPO-treated HFD mice also showed comparable LV mass (30.7±1.9 mg) to the control (32.7±1.8 mg, p=0.35; FIG. 5F). These results indicated mitochondrial oxidative stress might mediate diastolic dysfunction and LVH in obesity and type 2 DM.

### Mito-TEMPO Prevented Relaxation Impairment of Cardiomyocytes

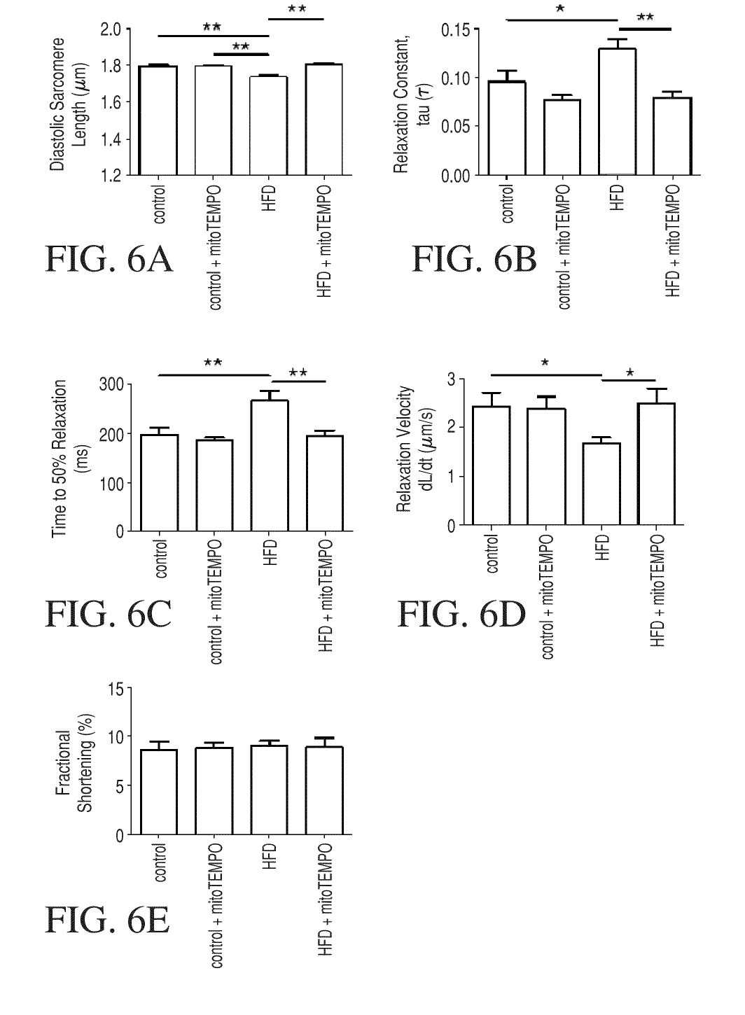

To assess diastolic dysfunction at the cellular level, sarcomeric contraction and relaxation was measured (FIGS. 6A-E). Diastolic sarcomere length was significantly shortened in the HFD mice (1.73±0.01 μm) compared with the control (1.80±0.01 μm, p<0.01). Mito-TEMPO treated mice showed comparable diastolic sarcomere length (1.81±0.01 μm, p=0.54) to the control. Relaxation i was also significantly increased in the HFD (0.13±0.01) and restored to the level of the control (0.10±0.01, p<0.05 vs. HFD) by mito-TEMPO treatment (0.08±0.01, p=0.22 vs. the control). Time to 50% relaxation was also significantly increased in the HFD mice (267±18 ms), compared with the control (197±15 ms, p<0.01). Mito-TEMPO treated mice showed similar time to 50% relaxation (195±11 ms) to the control (p=0.9). Relaxation velocity was significantly reduced in the HFD (1.66±0.13 μm/s) compared with the control (2.42±0.28 μm/s). Mito-TEMPO treated HFD mice showed similar relaxation velocity (2.48±0.3 μm/s, p=0.88) to the control. Finally, fractional shortening was similar throughout all these groups, indicating preserved systolic function in all groups. These results suggested that HFD mice developed diastolic dysfunction without systolic impairment at the cellular level, consistent with the in vivo imaging findings. Further, these results also indicated mitochondrial oxidative stress might be an underlying pathophysiology for relaxation impairment at the cellular level.

### Mito-TEMPO Reduced Mitochondrial Reactive Oxygen Species, Preserves Mitochondrial Ultrastructure and Prevents Oxidative Damage

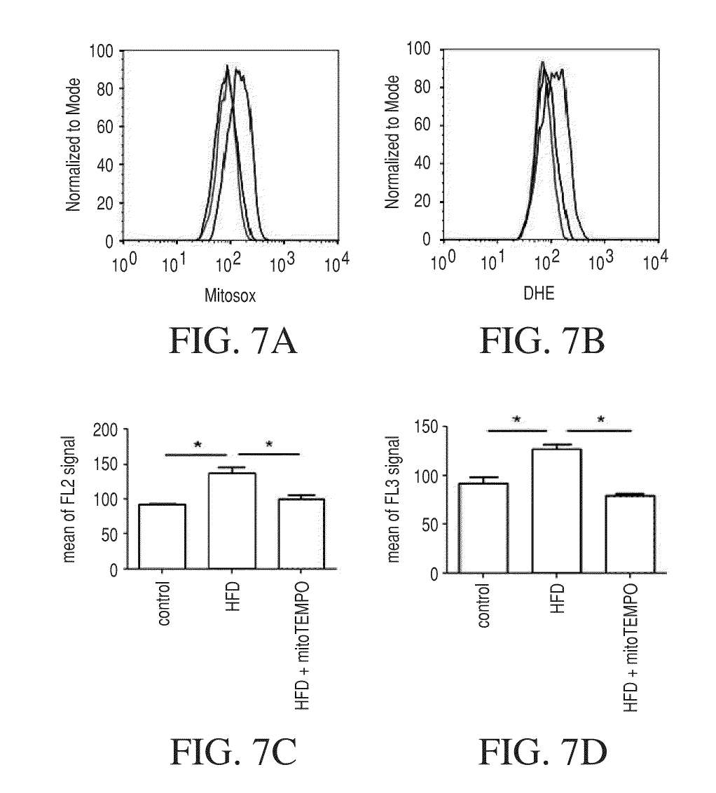

To quantify mitochondrial and cytosolic reactive oxygen species (ROS), primary cardiomyocytes from each group of mice were stained with mitoSOX and DHE. Then, the mean intensity was measured using the flow cytometry. HFD mice showed a significant increase in mitochondrial and cytosolic ROS compared to the control as evidenced by significant rightward shift of the histogram in FIGS. 7A-D. Quantitative assessment of the corresponding flow cytometry data showed a significant increase in the mitoSOX signal from the HFD (137±7) compared with the control (91±3, p<0.01). The mito-TEMPO treated group showed comparable mitoSOX signal (100±6, p=0.5) to the control, however. Confirming cellular oxidative stress, the DHE signal was also significantly increased in the HFD (127±5) compared with the control (97±8, p<0.01). Mito-TEMPO treated HFD mice showed similar DHE level (79.8±1.9) to the control (p=0.05), however.

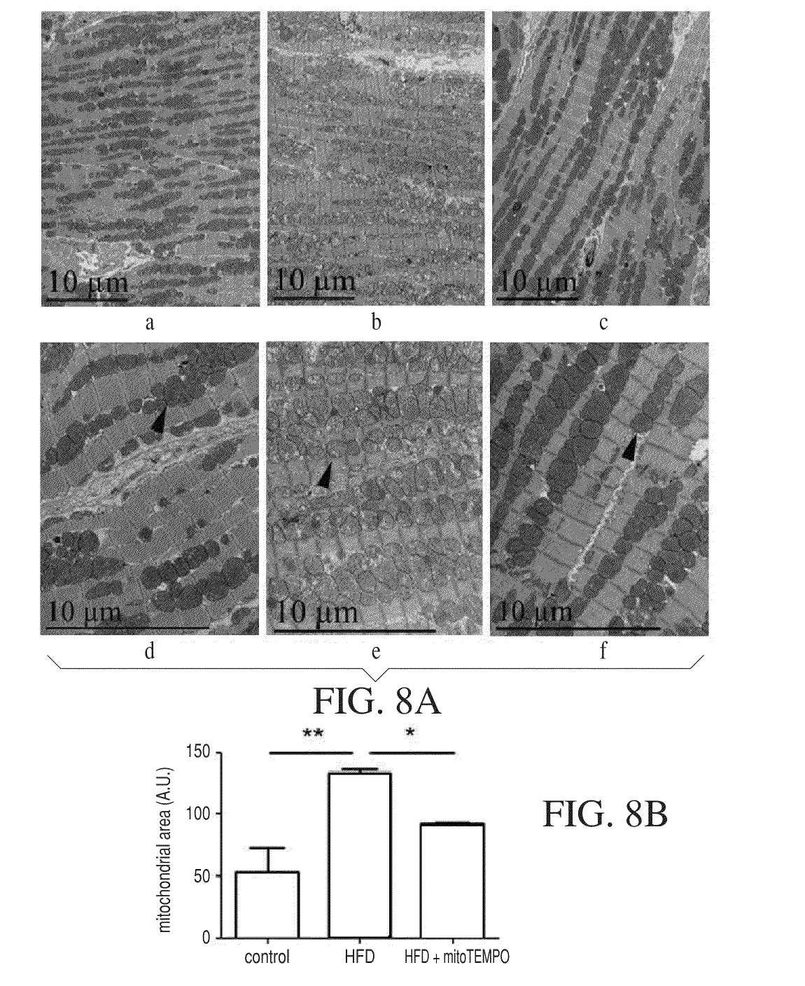

EM of animals on the HFD showed focal areas with evidence of mitochondrial swelling (FIGS. 8A and B). Mito-TEMPO treatment reversed these changes. There was evidence of mitochondrial proliferation in the HFD mice. Quantification of mitochondrial areas on a same magnification showed significant mitochondrial proliferation in the HFD (133.1±3.4) compared to the control (53.3±19, p<0.01). Mitochondrial areas in the mito-TEMPO treated mice (91.6±1.4) were comparable to the control (p=0.078) (FIG. 8B). These results indicated significant mitochondrial proliferation and increased biogenesis in HFD mice. Mitochondrial oxidative stress might mediate mitochondrial proliferation in obesity and type 2 DM.

### Mito-TEMPO Reduced ROS-Induced Protein Nitrotyrosylation in HFD Mice

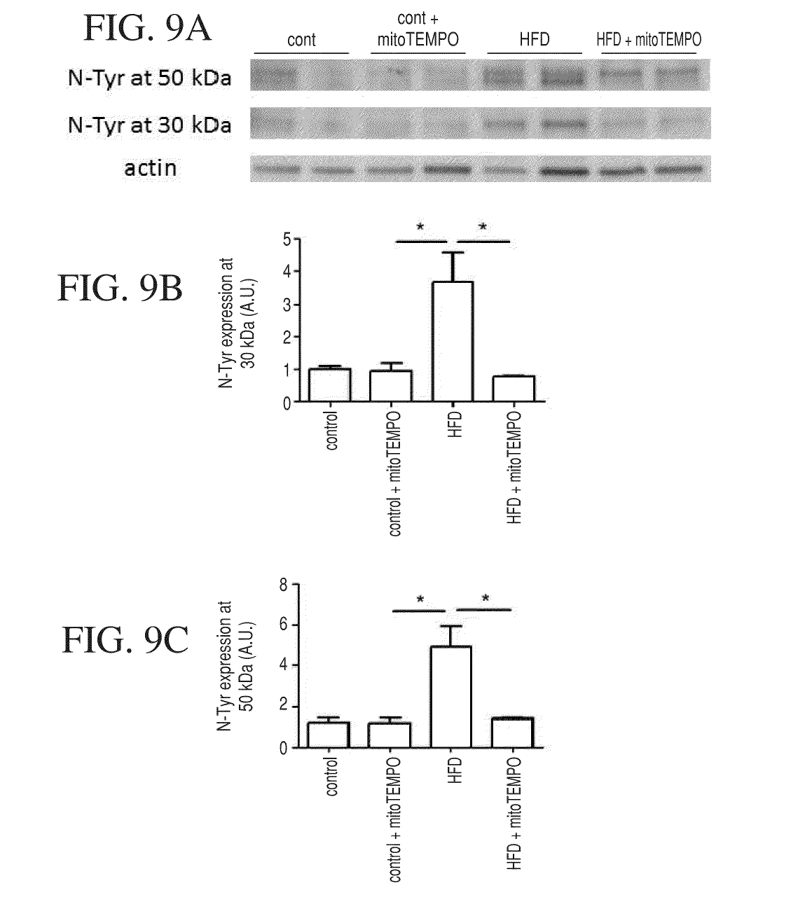

To quantify ROS-Induced protein modification, western blot was performed for nitrotyrosylation, as demonstrated in FIG. 9 A-C. Nitrotyrosylation of 30 kD and 50 kD proteins were significantly increased in the HFD mice compared with the control. With mito-TEMPO treatment, nitrotyrosylation was significantly reduced to the level of the control.

Mito-TEMPO Reduced ROS-Induced S-Glutathionylation of cMyBP-C in HFD Mice

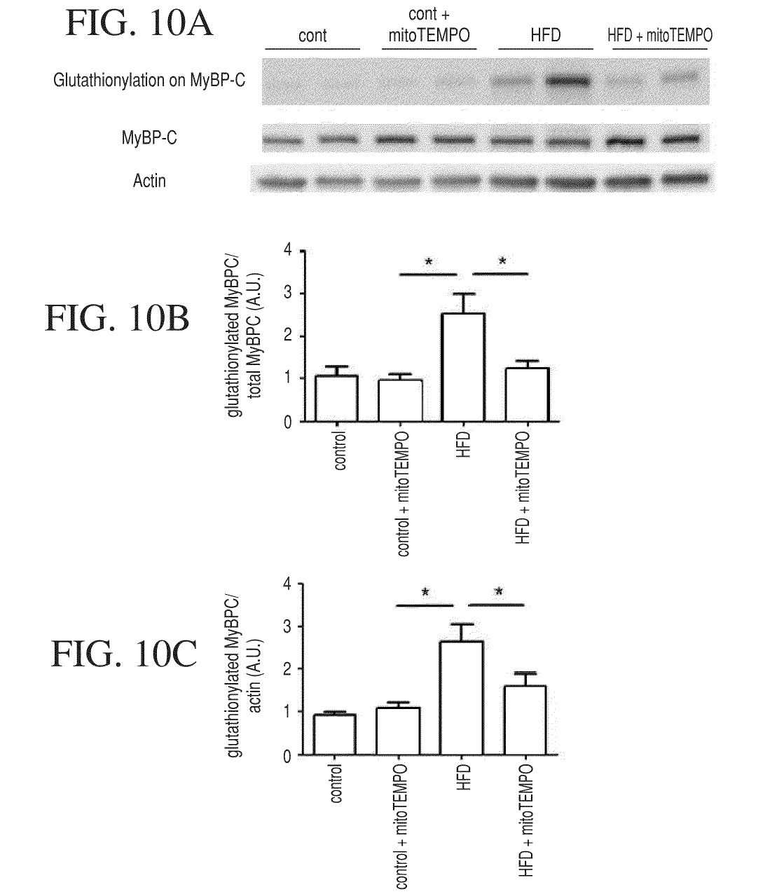

Previously we have shown glutathionylation on cMyBP-C leads to alteration in calcium sensitivity, resulting in diastolic dysfunction in the hypertensive mouse model. Similarly, as shown in FIGS. 10A-B, the HFD mice showed significantly increased S-glutathionylation on cMyBP-C compared to the control group. Mito-TEMPO treatment prevented this oxidant-mediated S-glutathionylation on cMyBP-C to the level of the control, as shown in FIGS. 9A-C. These results indicated mitochondrial oxidative stress highly associated with the elevation of S-glutathionylation on cMyBP-C and consequently increased myofilament calcium sensitivity, resulting in diastolic dysfunction.

### DISCUSSION

Here, we demonstrated HFD-induced insulin resistant mice developed obesity and diastolic dysfunction at the organ and cellular levels associated with mitochondrial oxidative stress and cMyBP-C S-glutathionylation. By treating these insulin resistant mice with a mitochondrial antioxidant, mito-TEMPO, insulin resistant were ameliorated as well as diastolic dysfunction in the heart. Myofilament contraction and relaxation regulating protein, cMyBP-C, were S-glutathionylated in HFD group, and mito-TEMPO was prevented its cMyBP-C S-gluatathionylation by reducing mitochondrial oxidative stress and regulated myocardial stiffness in diastolic function. This suggests that mitochondrial oxidative stress might mediate the epidemiological association of diastolic dysfunction and type 2 DM through alterations in cMyBP-C.

Epidemiological risk factors for diastolic dysfunction include obesity, type 2 DM, hypertension and age. Diastolic dysfunction is observed in about 40% of patients with type 2 DM. Previous reports indicated that left ventricular diastolic dysfunction represents the earliest preclinical manifestation of diabetic cardiomyopathy and that this can progress to symptomatic HF. Recent studies have demonstrated up to 60% of asymptomatic normoglycemic patients with type 2 DM have diastolic dysfunction when assessed by conventional echocardiography.

Recent studies show that insulin resistance is highly associated with reduced mitochondrial function followed by enhanced mitochondrial oxidative stress through mithochondrial superoxide dismutase (MnSOD) (Reference 18). Mitochondrial oxidative stress is upstream of insulin resistance suggesting that insulin resistance has a role in the antioxidant defense mechanism to protect cells from further oxidant damages (Reference 18).

The mechanism of diastolic dysfunction appears to be associated with glutathionylation of cMyBP-C. In the hypertensive mouse model, we found that diastolic dysfunction is not associated with changes in Ca²⁺ handling, fibrosis, titin isoform shifts, or increased AGE but is associated with glutathionylation of cMyBP-C. In a hypertensive mouse model, diastolic dysfunction is accompanied by cardiac oxidative stress, reduction in cardiac BH₄, and subsequent uncoupled NOS. Following unilateral nephrectomy and subcutaneous implantation of a controlled release DOCA pellet with 1% saline drinking water, C57 male mice develops mild hypertension and subsequent diastolic dysfunction in the absence of systolic dysfunction or cardiac hypertrophy, compared to sham-operated mice. These hypertensive mouse hearts show increased oxidized biopterins, NOS-dependent O₂^{•-} production, and reduced NO production. In the case of hypertension-associated DD, significant reduction in NO is developed because cardiac oxidative stress depletes the NOS co-factor BH₄, resulting in NOS uncoupling. Under this condition, electron flow from the reductase domain to the oxygenase domain is diverted to molecular oxygen rather than to L-arginine, leading to production of O₂^{•-} rather than NO. This O₂^{•-} production can be inhibited by the NOS inhibitor, N^{G}-nitro-L-arginine (L-NAME) and asymmetric dimethyl arginine (ADMA), so L-NAME suppressible O₂^{•-} production has become a marker of the presence of uncoupled NOS. Treatment of these hypertensive mice with BH₄ (5 mg/day) improves cardiac BH₄ storage and subsequently restores DD. Isolated cardiomyocytes from these hypertensive mice also show impaired relaxation, which was improved to the level of normal control cardiomyocytes with in-vitro BH₄ treatment (References 5-6). Targeted cardiac overexpression of angiotensin converting enzyme or heterozygous MnSOD knockout also demonstrates cardiac oxidative stress, NOS uncoupling, and diastolic dysfunction in the absence of hypertension. Similarly, our results demonstrated that HFD-induced insulin resistant mice showed significant mitochondrial oxidative stress and glutathionylation on cMyBP-C.

Although the treatment of diastolic dysfunction in type 2 DM has not been systematically studied in a large cohort, several small studies have shown conflicting results of potential benefit of anti-diabetic treatments (References 19-22).

These results suggest the underlying pathophysiologic mechanism of diastolic dysfunction in type 2 DM might not be directly related with glycemic control. As demonstrated in FIGS. 5A-F, fasting glucose level was similar in all the mouse groups, but fasting insulin level was significantly reduced in the control and mito-TEMPO treated HFD mice. Oxidative stress has been considered as a major common pathophysiology linking the type 2 DM and subsequent cardiac dysfunction. Approximately 90% ROS is produced by mitochondria in myocardial tissue (Reference 23).

Likewise, small animal studies have shown metallothionein overexpression or polyphenols resveratrol prevented diabetic mice from developing diastolic dysfunction and LVH through modification of mitochondrial oxidative stress (References 24-25).

In conclusion, we have demonstrated here that insulin resistant mice develop DD. This appears to be associated with mitochondrial oxidative stress. By treating these mice with mitochondrial specific anti-oxidant, diastolic function is preserved in the insulin resistant mice. In addition, insulin sensitivity was also maintained in these mito-TEMPO treated mice.

TABLE 1
Metabolic Characteristics

| | Control | HFD | p Value |
|---|---|---|---|
| Diet intake (g/day) | 4.5 ± 0.7 | 4.0 ± 0.4 | 0.53 |
| Body weight (g) | 27.5 ± 0.5 | 31 ± 0.6 | 0.001 |
| Body surface area | 89 ± 1 | 93 ± 1 | 0.03 |
| (cm²) | | | |
| Random serum | 223 ± 25 | 357 ± 23 | 0.003 |
| glucose (mg/dL) | | | |
| Fasting serum | 203 ± 30 | 239 ± 9 | 0.16 |
| glucose (mg/dL) | | | |
| Fasting serum | 1.2 ± 0.1 | 1.9 ± 0.1 | 0.003 |
| insulin (ng/mL) | | | |

TABLE 2
Cardiovascular Characteristics

| | Control | HFD | p Value |
|---|---|---|---|
| Systolic blood | 101 ± 3 | 107 ± 3 | 0.2 |
| pressure (mm Hg) | | | |
| Diastolic blood | 73 ± 3 | 75 ± 2 | 0.5 |
| pressure (mm Hg) | | | |
| Heart rate (bpm) | 518 ± 14 | 555 ± 8 | 0.02 |
| EDV (mm³) | 35.1 ± 1.4 | 30.3 ± 1.1 | 0.008 |
| ESV (mm³) | 11.2 ± 0.5 | 10.5 ± 0.3 | 0.26 |
| SV (mm³) | 23.1 ± 1.2 | 20.1 ± 1.0 | 0.036 |
| EF (%) | 67 ± 1 | 67 ± 0.8 | 0.93 |
| CO (mL/min) | 11.2 ± 0.4 | 12 ± 0.3 | 0.62 |
| Cardiac index(mL/min/m²) | 1.3 ± 0.05 | 1.3 ± 0.04 | 0.76 |
| LV mass (g) | 32.5 ± 1.2 | 39.3 ± 1 | 0.00 |
| LV mass index | 3.6 ± 0.1 | 4.2 ± 0.1 | 0.00 |
| (g/m²) | | | |

The invention also provides pharmaceutical or dietary supplemental compositions comprising 2-(2,2,6,6-Tetramethylpiperidin-1-oxyl-4-ylamino)-2-oxoethyl)triphenylphosphonium chloride (Mito-TEMPO). Accordingly, the compound (2-(2,2,6,6-Tetramethylpiperidin-1-oxyl-4-ylamino)-2-oxoethyl)triphenylphosphonium chloride (Mito-TEMPO)), can be formulated for oral or parenteral administration for the therapeutic or prophylactic treatment of diseases or conditions associated with and including diabetes, and diabetes-induced or related disorders, such as obesity, glucose intolerance, insulin resistance, and diastolic dysfunction, etc.

By way of illustration, the compound can be admixed with conventional pharmaceutical carriers and/or excipients and used in the form of tablets, capsules, elixirs, suspensions, syrups, wafers, and the like. Such pharmaceutical compositions contain from about 0.1 to about 90% by weight of the active compound (2-(2,2,6,6-Tetramethylpiperidin-1-oxyl-4-ylamino)-2-oxoethyl)triphenylphosphonium chloride (Mito-TEMPO)), and more generally from about 10 to about 30%. The pharmaceutical compositions may contain common carriers and excipients, such as corn starch, gelatin, lactose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

A liquid composition will generally consist of a suspension or solution of the compound or pharmaceutically acceptable salt in a suitable liquid carrier(s), for example ethanol, glycerine, sorbitol, non-aqueous solvent such as polyethylene glycol, oils or water, optionally with a suspending agent, a solubilizing agent (such as a cyclodextrin), preservative, surfactant, wetting agent, flavoring or coloring agent.

Alternatively, a liquid formulation can be prepared from a reconstitutable powder. For example a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example liquid paraffin or fractionated coconut oil can be prepared and filled into a soft gelatin capsule.

Tablet binders that can be included are acacia, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. Additionally, it may be desirable to add a coloring agent to make the dosage form more attractive in appearance or to help identify the product.

The compounds of the invention and their pharmaceutically acceptable salts that are active when given parenterally can be formulated for intramuscular, intrathecal, or intravenous administration. A typical composition for intramuscular or intrathecal administration consists of a suspension or solution of active ingredient in an oil, for example arachis oil or sesame oil. A typical composition for intravenous or intrathecal administration consists of a sterile isotonic aqueous solution containing, for example active ingredient and dextrose or sodium chloride, or a mixture of dextrose and sodium chloride. Other examples of aqueous solution are lactated Ringers injection, lactated Ringer's plus dextrose injection, Normosol-M and dextrose, Isolyte E, acylated Ringer's injection, and the like. Optionally, a co-solvent, for example, polyethylene glycol; a chelating agent, for example, ethylenediamine tetracetic acid; a solubilizing agent, for example, a cyclodextrin; and an anti-oxidant, for example, sodium metabisulphite, may be included in the formulation. Alternatively, the solution can be freeze dried and then reconstituted with a suitable solvent just prior to administration.

The compounds of the invention which are active on rectal administration can be formulated as suppositories. A typical suppository formulation will generally consist of active ingredient with a binding and/or lubricating agent such as a gelatin or cocoa butter or other low melting vegetable or synthetic wax or fat.

The active compound is effective over a wide dosage range and is generally administered in a therapeutically effective amount. It, will be understood, however, that the amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like. Suitable doses are selected to effect a blood concentration of about 100-300 μM, preferably 100 μM.

According to the invention, a compound can be administered in a single daily dose or in multiple doses per day. The treatment regimen may require administration over extended periods of time, for example, for several days, for from one to six weeks, or longer.

Suitable formulations for use in the present invention can be found in Remington's Pharmaceutical Sciences, Mace Publishing Company, Philadelphia, Pa., 17th ed. (1985).

The compositions of the present invention can be used to treat conditions associated with and including diabetes and related disorders, such as obesity, glucose intolerance, insulin resistance, diastolic dysfunction, etc., including all disease states and/or conditions that are acknowledged now, or that are found in the future, to be associated with the activity of sugar level, insulin level, obesity, ROS level, etc. Such disease states include, but are not limited to diabetes, obesity, glucose intolerance, insulin resistance, diastolic dysfunction, pathophysiological disorders, including hypertension, cardiac arrhythmogenesis, sudden cardiac death (SCD), ventricular tachycardia (VT), insulin-dependent diabetes, non-insulin dependent diabetes mellitus, diabetic neuropathy, seizures, tachycardia, ischemic heart disease, cardiac failure, angina, myocardial infarction, ventricular fibrillation, transplant rejection, autoimmune disease, sickle cell anemia, muscular dystrophy, gastrointestinal disease, mental disorder, sleep disorder, anxiety disorder, eating disorder, neurosis, alcoholism, inflammation, cerebrovascular ischemia, CNS diseases, epilepsy, Parkinson's disease, asthma, incontinence, urinary dysfunction, micturition disorder, irritable bowel syndrome, restenosis, subarachnoid hemorrhage, Alzheimer disease, drug dependence/addiction, schizophrenia, Huntington's chorea, tension-type headache, trigeminal neuralgia, cluster headache, migraine (acute and prophylaxis), inflammatory pain, neuropathic pain and depression.

While this invention has been described as having preferred sequences, ranges, steps, order of steps, materials, structures, shapes, configurations, features, components, or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the appended claims.

### REFERENCES

The following references, and those cited in the disclosure herein, are hereby incorporated herein in their entirety by reference.
(1) Schocken D D, Benjamin E J, Fonarow G C et al. Prevention of heart failure: a scientific statement from the American Heart Association Councils on Epidemiology and Prevention, Clinical Cardiology, Cardiovascular Nursing, and High Blood Pressure Research; Quality of Care and Outcomes Research Interdisciplinary Working Group; and Functional Genomics and Translational Biology Interdisciplinary Working Group. Circulation 2008 May 13; 117(19):2544-65.
(2) Owan T E, Hodge D O, Herges R M, Jacobsen S J, Roger V L, Redfield M M. Trends in prevalence and outcome of heart failure with preserved ejection fraction. N Engl J Med 2006 Jul. 20; 355(3):251-9.
(3) Redfield M M, Jacobsen S J, Burnett J C, Jr., Mahoney D W, Bailey K R, Rodeheffer R J. Burden of systolic and diastolic ventricular dysfunction in the community: appreciating the scope of the heart failure epidemic. JAMA 2003 Jan. 8; 289(2):194-202.
(4) Patil V C, Patil H V, Shah K B, Vasani J D, Shetty P. Diastolic dysfunction in asymptomatic type 2 diabetes mellitus with normal systolic function. J Cardiovasc Dis Res 2011 October; 2(4):213-22.
(5) Silberman G A, Fan T H, Liu H et al. Uncoupled cardiac nitric oxide synthase mediates diastolic dysfunction. Circulation 2010 Feb. 2; 121 (4):519-28.
(6) Jeong E M, Monasky M M, Gu L et al. Tetrahydrobiopterin improves diastolic dysfunction by reversing changes in myofilament properties. J Mol Cell Cardiol 2013 March; 56:44-54.
(7) Baynes J W. Role of oxidative stress in development of complications in diabetes. Diabetes 1991 April; 40(4):405-12.
(8) Baynes J W, Thorpe S R. Role of oxidative stress in diabetic complications: a new perspective on an old paradigm. Diabetes 1999 January; 48(1):1-9.
(9) Saxena A K, Srivastava P, Kale R K, Baquer N Z. Impaired antioxidant status in diabetic rat liver. Effect of vanadate. Biochem Pharmacol 1993 Feb. 9; 45(3):539-42.
(10) Hankiewicz J H, Banke N H, Farjah M, Lewandowski E D. Early impairment of transmural principal strains in the left ventricular wall after short-term, high-fat feeding of mice predisposed to cardiac steatosis. Circ Cardiovasc Imaging 2010 November; 3(6):710-7.
(11) Hankiewicz J H, Goldspink P H, Buttrick P M, Lewandowski E D. Principal strain changes precede ventricular wall thinning during transition to heart failure in a mouse model of dilated cardiomyopathy. Am J Physiol Heart Circ Physiol 2008 January; 294(1):H330-H336.
(12) Liu W, Chen J, Ji S et al. Harmonic phase MR tagging for direct quantification of Lagrangian strain in rat hearts after myocardial infarction. Magn Reson Med 2004 December; 52(6):1282-90.
(13) Fogel M A, Weinberg P M, Hubbard A, Haselgrove J. Diastolic biomechanics in normal infants utilizing MRI tissue tagging. Circulation 2000 Jul. 11; 102(2):218-24.
(14) Edvardsen T, Rosen B D, Pan L et al. Regional diastolic dysfunction in individuals with left ventricular hypertrophy measured by tagged magnetic resonance imaging—the Multi-Ethnic Study of Atherosclerosis (MESA). Am Heart J 2006 January;151(1):109-14.
(15) Osman N F, Kerwin W S, McVeigh E R, Prince J L. Cardiac motion tracking using CINE harmonic phase (HARP) magnetic resonance imaging. Magn Reson Med 1999 December; 42(6):1048-60.
(16) Lovelock J D, Monasky M M, Jeong E M et al. Ranolazine improves cardiac diastolic dysfunction through modulation of myofilament calcium sensitivity. Circ Res 2012 Mar. 16; 110(6):841-50.
(17) Sovari A A, Rutledge C A, Jeong E M et al. Mitochondria oxidative stress, connexin 43 remodeling, and sudden arrhythmic death. Circ Arrhythm Electrophysiol 2013 June; 6(3):623-31.
(18) Hoehn K L, Salmon A B, Hohnen-Behrens C et al. Insulin resistance is a cellular antioxidant defense mechanism. Proc Natl Acad Sci U S A 2009 Oct. 20; 106(42):17787-92.
(19) von BH, Diamant M, Scheffer P G, Siegmund T, Schumm-Draeger P M. Rosiglitazone, but not glimepiride, improves myocardial diastolic function in association with reduction in oxidative stress in type 2 diabetic patients without overt heart disease. Diab Vasc Dis Res 2008 November; 5(4):310-8.
(20) St John S M, Rendell M, Dandona P et al. A comparison of the effects of rosiglitazone and glyburide on cardiovascular function and glycemic control in patients with type 2 diabetes. Diabetes Care 2002 November; 25(11):2058-64.
(21) Lautamaki R, Airaksinen K E, Seppanen M et al. Rosiglitazone improves myocardial glucose uptake in patients with type 2 diabetes and coronary artery disease: a 16-week randomized, double-blind, placebo-controlled study. Diabetes 2005 September; 54(9):2787-94.
(22) van der Meer R W, Rijzewijk L J, de Jong H W et al. Pioglitazone improves cardiac function and alters myocardial substrate metabolism without affecting cardiac triglyceride accumulation and high-energy phosphate metabolism in patients with well-controlled type 2 diabetes mellitus. Circulation 2009 Apr. 21; 119(15):2069-77.
(23) Raha S, Robinson B H. Mitochondria, oxygen free radicals, disease and ageing. Trends Biochem Sci 2000 October; 25(10):502-8.
(24) Qin F, Siwik D A, Luptak I et al. The polyphenols resveratrol and S17834 prevent the structural and functional sequelae of diet-induced metabolic heart disease in mice. Circulation 2012 Apr. 10; 125(14):1757-6.
(25) Zhou G, Li X, Hein D W et al. Metallothionein suppresses angiotensin Il-induced nicotinamide adenine dinucleotide phosphate oxidase activation, nitrosative stress, apoptosis, and pathological remodeling in the diabetic heart. J Am Coll Cardiol 2008 Aug. 19; 52(8):655-66.

What is claimed is:

1. A method of treating, reversing, or ameliorating type 2 diabetes (DM), comprising:
administering to a host in need thereof a therapeutically effective amount of a mitochondria-targeted antioxidant.

2. The method of claim 2, wherein:
the antioxidant comprises mito-TEMPO (2-(2,2,6,6-Tetramethylpiperidin-1-oxyl-4-ylamino)-2-oxoethyl)triphenylphosphonium chloride).

3. The method of claim 2, wherein the step of administering comprises:
administering mito-TEMPO in at least one form selected from the group consisting of a dietary supplement, a composition, a pharmaceutical composition, and a combination thereof.

4. The method of claim 2, wherein the step of administering comprises:
administering mito-TEMPO orally or intravenously.

5. The method of claim 2, wherein the host is a human.

6. The method of claim 2, wherein the host is an animal.

7. A method of treating, reversing, or ameliorating insulin resistance, comprising:
administering to a host in need thereof a therapeutically effective amount of a mitochondria-targeted antioxidant.

8. The method of claim 7, wherein:
the antioxidant comprises mito-TEMPO (2-(2,2,6,6-Tetramethylpiperidin-1-oxyl-4-ylamino)-2-oxoethyl)triphenylphosphonium chloride).

9. The method of claim 8, wherein the step of administering comprises:
administering mito-TEMPO in at least one form selected from the group consisting of a dietary supplement, a composition, a pharmaceutical composition, and a combination thereof.

10. The method of claim 8, wherein the step of administering comprises:
administering mito-TEMPO orally or intravenously.

11. The method of claim 8, wherein the host is a human.

12. The method of claim 8, wherein the host is an animal.

13. A method of treating, reversing, or ameliorating glucose intolerance, comprising:
administering to a host in need thereof a therapeutically effective amount of a mitochondria-targeted antioxidant.

14. The method of claim 13, wherein:
the antioxidant comprises mito-TEMPO (2-(2,2,6,6-Tetramethylpiperidin-1-oxyl-4-ylamino)-2-oxoethyl)triphenylphosphonium chloride).

15. The method of claim 13, wherein the step of administering comprises:
administering mito-TEMPO in at least one form selected from the group consisting of a dietary supplement, a composition, a pharmaceutical composition, and a combination thereof.

16. The method of claim 13, wherein the step of administering comprises:
administering mito-TEMPO orally or intravenously.

17. The method of claim 13, wherein the host is a human.

18. The method of claim 13, wherein the host is an animal.

19. A method of treating, reversing, or ameliorating diabetes-induced diastolic dysfunction, comprising:
reducing S-glutathionylated myosin binding protein-C (MyBP-C) level by administering to a host in need thereof a therapeutically effective amount of a mitochondria-targeted antioxidant.

20. The method of claim 19, wherein:
the antioxidant comprises mito-TEMPO (2-(2,2,6,6-Tetramethylpiperidin-1-oxyl-4-ylamino)-2-oxoethyl)triphenylphosphonium chloride).

21. The method of claim 20, wherein the step of administering comprises:
administering mito-TEMPO in at least one form selected from the group consisting of a dietary supplement, a composition, a pharmaceutical composition, and a combination thereof.

22. The method of claim 20, wherein the step of administering comprises:
administering mito-TEMPO orally or intravenously.

23. The method of claim 20, wherein the host is a human.

24. The method of claim 20, wherein the host is an animal.
